(19) United States
(12) Patent Application Publication
(10) Pub. No.: US 2024/0299382 A1
(43) Pub. Date: Sep. 12, 2024

(54) COMPOUNDS AND METHODS FOR DEGRADING RCOR1, LSD1, HDAC1 AND HDAC2 IN THE COREST

(71) Applicant: University of Florida Research Foundation, Inc., Gainesville, FL (US)

(72) Inventor: Daiqing LIAO, Gainesville, FL (US)

(21) Appl. No.: 18/572,491

(22) PCT Filed: Jun. 27, 2022
(86) PCT No.: PCT/US2022/073187

§ 371 Date: Dec. 20, 2023

Related U.S. Application Data
(60) Provisional application No. 63/216,096, filed on Jun. 29, 2021.

(51) Int. Cl. A61K 31/505; A61K 31/166; A61K 31/24; A61P 35/00

(52) U.S. Cl. CPC A61K 31/505; A61K 31/166; A61K 31/24; A61P 35/00

(57) ABSTRACT

Described herein are compounds methods for reducing the amount of rest compressor 1 (RCOR1, also known as CoREST1), lysine-specific histone demethylase 1A (LSD1, also known as KDM1A), histone deacetylase 1 (HDAC1), histone deacetylase 2 (HDAC2) or any combination thereof in a subject. These proteins are core subunits of the CoREST complex that has both lysine deacetylase and lysine demethylase activity. By reducing the amount of RCOR1, LSD1, HDAC1 and/or HDAC2, cancer cell proliferation and tumor growth are impaired. In one aspect, the compounds useful herein have the formula I or the pharmaceutically acceptable salt thereof.

### CROSS-REFERENCE TO RELATED APPLICATIONS

This application claims the benefit of and priority to co-pending U.S. Provisional Patent Application No. 63/216,096, filed on Jun. 29, 2021, the contents of which are incorporated by reference herein in their entireties.

### STATEMENT REGARDING SPONSORED RESEARCH OR DEVELOPMENT

This invention was made in whole or in part from funding received under grant numbers 20K07, 21K03, 22K05, 6BC03 and 6JK03, awarded by the Florida Department of Health.

### BACKGROUND

Breast cancer (BC) is the most frequently diagnosed cancer and the second leading cause of cancer-related death in women. According to the American Cancer Society, there are more than 3.1 million BC survivors (women still being treated and those who have completed treatment), and there will be 279, 100 new cases of invasive BC diagnoses in 2020 in the US. It was estimated that there are 1.7 million new BC diagnoses per year worldwide.

BC is classified into four major subtypes including estrogen receptor positive (ER+) luminal (A and B), HER2-enriched subtypes and basal/triple negative BC (TNBC) lacking hormone receptor expression and HER2 gene amplification. Endocrine therapies (ETs, e.g. tamoxifen) that block estrogen-induced ER activation are standard therapies for treating ER+ BC.¹ While ETs have significantly prolonged the survival of many patients with ER+ BC,² it was estimated that nearly 50% ER+ BC fail to respond to such therapies, and ER+ but ET-resistant BC generally has poor prognosis.^{1,3-5} For the HER2-enriched subtype, therapy that blocks HER2 signaling such as trastuzumab and lapatinib is commonly used. Unfortunately, however, only about 35% of HER2-enriched tumors show response to HER2-targeted therapies and acquired resistance to these therapies is a major hurdle for sustained treatment response.⁶ TNBC accounts for 15 to 20% of all BC cases. Chemotherapy remains the mainstay treatments with TNBC. However, residual disease and relapse after chemotherapy are common among TNBC patients.⁷⁻¹¹ Therefore, current standard therapies are inadequate to meet the medical needs of many patients with BC due to de novo or acquired resistance. Novel effective treatment regimens are clearly needed to improve clinical outcome for patients with advanced BC.

### SUMMARY

Described herein are compounds methods for reducing the amount of rest compressor 1 (RCOR1, also known as CoREST1), lysine-specific histone demethylase 1A (LSD1, also known as KDM1A), histone deacetylase 1 (HDAC1), histone deacetylase 2 (HDAC2) or any combination thereof in a subject. These proteins are core subunits of the CoREST complex that has both lysine deacetylase and lysine demethylase activity. By reducing the amount of RCOR1, LSD1, HDAC1 and/or HDAC2, cancer cell proliferation and tumor growth are impaired. In one aspect, the compounds of formula I or the pharmaceutically acceptable salt thereof

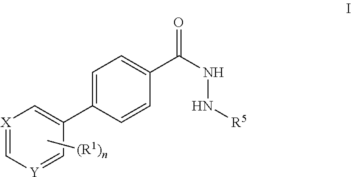

wherein X and Y are independently N or CR², wherein R¹ and R² are independently hydrogen, an alkyl group, a halo group, or a carbonyl group, R⁵ is a C₁ to C₁₀ alkyl group, and n is an integer from 0 to 3.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-B show SR-4370 analogs exhibit molecular glue function to degrade RCOR1, LSD1, HDAC1 and HDAC2 in a neddylation and proteasome-dependent manner.

FIG. 2 shows SR-4370 specifically and potently degrades RCOR1, LSD1, HDAC1 and HDAC2 in a neddylation and proteasome-dependent manner.

FIGS. 3A-C show SR-4370 is efficacious against breast cancer in vivo.

FIG. 4 shows model depicting how SR-4370 recruits a CRL to the CoREST complex for ubiquitination.

FIGS. 5A-B show SR-4370 is more potent than UF010 to impair breast cancer cell growth.

FIG. 6 shows SR-4370 is safe as judged by body weights during treatment. Female BALB/c mice bearing the EMT6 tumors were dosed with vehicle or SR-4370 (50 mg/kg) via intraperitoneal injection. Mouse body weights are shown during the treatment period.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a solvent” include, but are not limited to, mixtures or combinations of two or more such solvents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The terms “halo,” “halogen” or “halide,” as used herein can be used interchangeably and refer to F, Cl, Br, or I.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human).

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of a cancer, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

As used herein rest compressor 1 (RCOR1) also known as CoREST1 is a protein that in humans is encoded by the RCOR1 gene.

As used herein, lysine-specific histone demethylase 1A (LSD1) also known as lysine (K)-specific demethylase 1A (KDM1A) is a protein in humans that is encoded by the KDM1A gene.

As used herein, histone deacetylase 1 (HDAC1) is a protein in humans that is encoded by the HDAC1 gene.

As used herein, histone deacetylase 2 (HDAC2) is a protein in humans that is encoded by the HDAC2 gene.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to conduct the methods of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Methods for Reducing the Amount of RCOR1, LSD1, HDAC1 and/or HDAC2

HDACs are critical regulators of cancer epigenomes. Among all HDACs, class I HDACs (HDACs 1-3 and 8) are critical to activating oncogenes underlying tumorigenesis, disease progression and treatment resistance.¹²⁻¹⁵ Recent studies have shown that HDACs 1-3 are important for oncogene expression regulated by super enhancers in breast and other cancer types.^{16,17} HDAC1 and 2 (HDAC1/2) are close paralogs with a high degree of sequence identity. Their function is generally considered exchangeable, and both can be assembled into the same protein complex as a heterodimer, such as when they are incorporated into the CoREST complex.¹⁸ The expression levels of HDAC1/2 are significantly increased in all breast cancer (BC) subtypes, especially in basal/TNBC subtype, and appear to be further increased in BC metastases. Notably, high levels of HDAC2 correlate with poor patient survival and treatment resistance. In HDACi clinical trials, it was found that HDAC2 expression correlates with treatment response to HDACi.¹⁹⁻²¹

HDAC1/2 are assembled into multi-subunit protein complexes such as the CoREST, NuRD, SIN3 and MiDAC complexes.²² The CoREST complex is unique in that it has a deacetylase subunit (HDAC1 or 2) and a demethylase subunit (LSD1/KDAM1A).²³⁻²⁵ LSD1 is a flavin-dependent amine oxidase that demethylates mono- or di-methylated Lys4 in histone H3 (H3K4me1/2).²⁴ More recently, it was reported that an LSD1 isozyme called LSD1+8a can demethylate H3K9me2.²⁶ One of three RCOR proteins (RCOR1, 2 and 3, also known as CoREST1, 2 and 3) serves as a scaffold that binds directly to both HDAC1/2 and LSD1. The deacetylase and demethylase activity may act in concert for efficient histone deacetylation and demethylation in chromatin.^{25,27} Because chromatin with acetylated lysines and methylated H3K4 modifications are typically associated with gene activation, the enzymatic activities of HDAC1/2 and LSD1 in the CoREST complex seem to operate synergistically for gene silencing,^{28,29} although recent studies have shown that histone deacetylation and LSD1-mediated demethylation are also important for activating gene expression.^{17,26,30-32} LSD1 is markedly upregulated in all BC subtypes, especially in Basal/TN subtype. LSD1 displays oncogenic function in BC. It confers stem cell-like traits to BC cells and chemoresistance,^{33,34} and may also play a role in resistance to cancer immunotherapy.³⁵ Significantly, LSD1 overexpression correlates with poor prognosis in basal/TNBC.³⁶ RCOR1 also appears to have oncogenic function in BC and other cancer types.^{37,38}

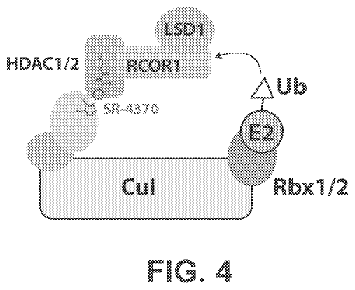

Described herein are compounds and methods for reducing the amount of rest compressor 1 (RCOR1), lysine-specific histone demethylase 1A (LSD1), HDAC1, HDAC2 or a combination thereof in a subject. By reducing the amount of RCOR1, LSD1, HDAC1 and/or HDAC2, cancer cell proliferation and tumor growth are impaired. In one aspect, the downregulation of RCOR1, LSD1, HDAC1, and HDAC2 can be completely reversed by inhibition of Cullin neddylation (MLN4924³⁹) and inhibition of the proteasome (MG132). The compounds described herein can trigger RCOR1, LSD1, HDAC1, and HDAC2 degradation through a Cullin-RING E3 ubiquitin ligase (CRL)-mediated ubiquitination and the proteasome. Thus, the compounds described herein are class of molecular glue-like degraders. Not wishing to be bound by theory, recruits a CRL to ubiquitinate RCOR1, LSD1, HDAC1 and HDAC2 in the CoREST complex (FIG. 4), resulting in their proteasomal degradation. RCOR1, LSD1, HDAC1 and HDAC2 degradation results in epigenomic reprogramming and impairs cancer cell proliferation and tumor growth.

In one aspect, the compounds described herein can be administered to a subject that has been diagnosed with cancer associated with elevated levels of RCOR1, LSD1, HDAC1 and HDAC2⁴⁰, which include breast cancer^{34,36,37,41-44}, prostate cancer^{45,46}, colon cancer³⁸, lung cancer^{47,48}, liver cancer 49, brain tumors, melanomas²⁸, Merkel cell carcinomas⁵⁰, leukemias and lymphomas⁵¹⁻⁵⁷, ovarian cancers⁵⁸, and pediatric cancer such as sarcomas, neuroblastomas, and gliomas^{59,60}. In one aspect, the subject has breast cancer of various subtypes including hormone receptor positive, HER2-enriched and TNBC^{61,62}.

LSD1 demethylates the pioneer transcription factor FOXA1 and stimulates its chromatin-binding⁶³. This facilitates chromatin-binding of steroid hormone receptors such as the androgen receptor and estrogen receptors. The androgen receptor and estrogen receptor are dominant drivers of hormone-dependent prostate and breast cancer respectively. Therefore, LSD1 inhibition can be used to block the FOXA1-steroid hormone receptor pathways for the treatment hormone-dependent prostate and breast cancer.

LSD1 inhibition reactivates endogenous retroviruses, provoking antitumor immune response⁶⁴. LSD1, HDAC1 and HDAC2 inhibitors have been shown to enhance the anticancer efficacy of cancer immunotherapy^{35,65,66}.

LSD1 has been shown to suppress autophagy⁶⁷. Pharmacological or genetic inhibition of LSD1 induces autophagy. Pharmacological induction of autophagy can be used to treat liver fibrosis, infectious diseases, and neurodegenerative diseases such as Alzheimer's disease. LSD1 is also mechanistically linked to fatty liver disease and type 2 diabetes and LSD1 inhibitors such as by using the compounds described herein can be used to treat these diseases⁶⁸.

LSD1 promotes inflammation through stabilization of the p65 subunit of the NF-κB complex. LSD1 inhibition attenuates sepsis-induced mortality^{69,70}. LSD1 inhibition has also been shown to potentially attenuate SARS-CoV-2-triggered cytokine release syndrome⁷¹. Therefore pharmacological inhibition of LSD1 such as by using the compounds described herein can be used to treat inflammatory diseases such as sepsis, rheumatoid arthritis, and COVID19.

In one aspect, the amount of RCOR1 and/or LSD1 reduced in the subject by the compounds described herein is at least two times the amount when compared to the levels of RCOR1 and/or LSD1 in the same prior to administration of the compound. In another aspect, the amount of RCOR1 and/or LSD1 reduced in the subject by the compounds described herein is at least two times, three times, four times, or five times the amount when compared to the levels of RCOR1 and/or LSD1 in the same prior to administration of the compound.

In one aspect, HDAC1 and HDAC2 is notably reduced in the subject by the compounds described herein when compared to the levels of RCOR1 and/or LSD1 in the same prior to administration of the compound.

In one aspect, the amount of rest compressor 1 (RCOR1), lysine-specific histone demethylase 1A (LSD1), HDAC1, HDAC2 or a combination thereof is measured in a subject prior to the administration of the compound. If the amount of rest compressor 1 (RCOR1), lysine-specific histone demethylase 1A (LSD1), HDAC1 and/or HDAC2 is above a certain level, one or more compounds described herein can be administered. The amount of the compound to be administered can vary depending upon the amount of rest compressor 1 (RCOR1), lysine-specific histone demethylase 1A (LSD1), HDAC1 and/or HDAC2 present in the subject.

In another aspect, the SR-4370 and analogs are found to selectively degrade HDAC1 and HDAC2 among different HDAC isozymes. HDAC1 and HDAC2 overexpression has been associated with diverse diseases including various types of cancer, cardiovascular disease, inflammatory diseases, metabolic diseases such as diabetes, neurological and neurodegenerative disorders and infectious diseases. Therefore, the SR-4370 and analogs are applicable to treating these diverse diseases wherein overexpression or hyperactivity of HDAC1 and HDAC2 are pertinent to the etiology and progression of these diseases.

### Compounds

In one aspect, compounds used herein have formula I

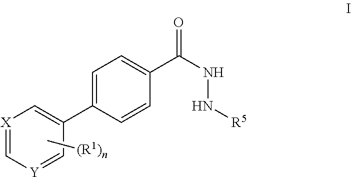

wherein X and Y are independently N or CR²,
wherein R¹ and R² are independently hydrogen, an alkyl group, a halo group, or an ester group,
R⁵ is a C₁ to C₁₀ alkyl group, and
n is an integer from 0 to 3.

In one aspect, X and Y in formula I are each N. In another aspect, X and Y in formula I are each N, and n is 0.

In one aspect, X and Y in formula I are each CR². In one aspect, X in formula I is CF, CH, or C—C(O)OR³, wherein R³ is an alkyl group. In another aspect, X in formula I is CF, CH, or C—C(O)OR³, wherein R³ is an alkyl group, and n is 0.

In one aspect, Y in formula I is CH or CR⁴, wherein R⁴ is an alkyl group. In one aspect, Y in formula I is CH or CR⁴, wherein R⁴ is an alkyl group, and n is 0. In another aspect, X is CF and Y is CH or CR⁴, wherein R⁴ is an alkyl group. In another aspect, X is CF, Y is CH, R¹ is fluoro, and n is 1.

The method of claim 1, wherein the compound has the formula II

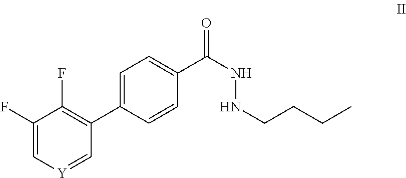

wherein Y is CH or CR⁴, wherein R⁴ is an alkyl group.

In one aspect, the compound has the formula III

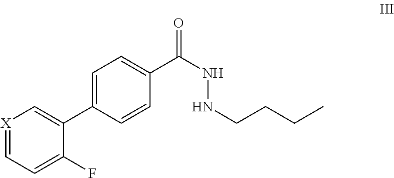

wherein X is C—C(O)OR³, wherein R³ is an alkyl group.

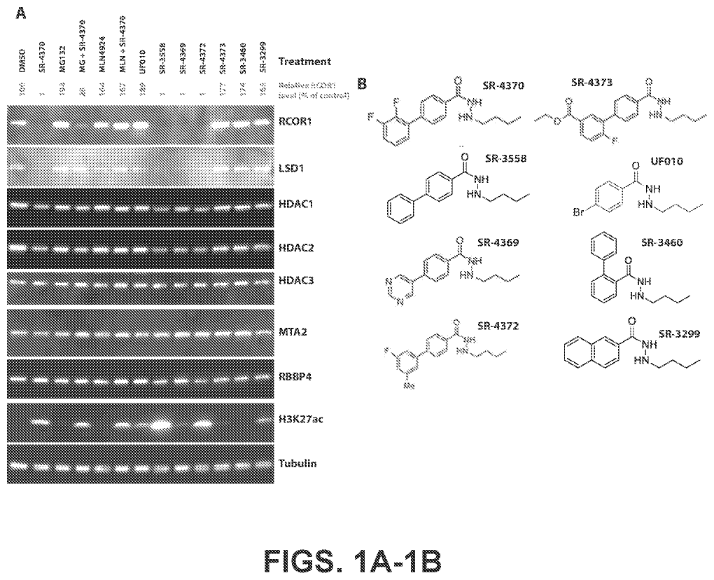

In another aspect, the compound is SR-4370, SR-3558, SR-4369, SR-4372, or SR-4373 as provided in FIG. 1A. The compounds described herein can be synthesized using the procedures provided in U.S. Pat. No. 10,897,944, which is incorporated by reference in its entirety.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentaerythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N, N-diethyl acetamide, N, N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

### Aspects

The present disclosure can be described in accordance with the following numbered Aspects, which should not be confused with the claims.

Aspect 1: A method for reducing the amount of rest compressor 1 (RCOR1), lysine-specific histone demethylase 1A (LSD1), histone deacetylase 1 (HDAC1), histone deacetylase 2 (HDAC2), or any combination thereof in a subject comprising administering to the subject a compound of formula I or the pharmaceutically acceptable salt thereof

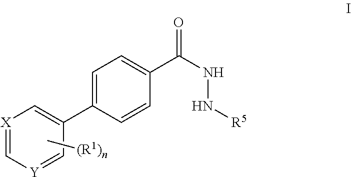

wherein X and Y are independently N or CR²,
wherein R¹ and R² are independently hydrogen, an alkyl group, a halo group, or an ester group,
R⁵ is a C₁ to C₁₀ alkyl group; and
n is an integer from 0 to 3.

Aspect 2: The method of Aspect 1, wherein X and Y are each N.

Aspect 3: The method of Aspect 2, wherein n is 0.

Aspect 4: The method of Aspect 1 or 2, wherein R⁵ is a butyl group.

Aspect 5: The method of Aspect 1, wherein X and Y are each CR².

Aspect 6: The method of Aspect 1, wherein X is CF.

Aspect 7: The method of Aspect 1, wherein X is CH.

Aspect 8: The method of Aspect 1, wherein X is C—C(O)OR³, wherein R³ is an alkyl group.

Aspect 9: The method of any one of Aspects 5-8, wherein Y is CH.

Aspect 10: The method of any one of Aspects 5-8, wherein Y is CR⁴, wherein R⁴ is an alkyl group.

Aspect 11: The method of any one of Aspects 5-10, wherein n is 0.

Aspect 12: The method of any one of Aspects 5-11, wherein R⁵ is a butyl group.

Aspect 13: The method of Aspect 1, wherein X is CF and Y is CH or CR⁴, wherein R⁴ is an alkyl group.

Aspect 14: The method of Aspect 1, wherein the compound has the formula II

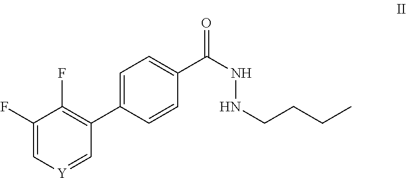

wherein Y is CH or CR⁴, wherein R⁴ is an alkyl group.

Aspect 15: The method of Aspect 1, wherein X is CF, Y is CH, R¹ is fluoro, and n is 1.

Aspect 16: The wherein the compound has the formula III

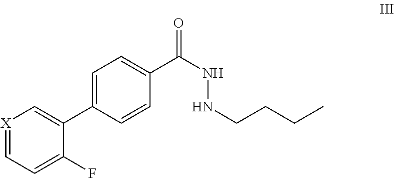

wherein X is C—C(O)OR³, wherein R³ is an alkyl group.

Aspect 17: The method of Aspect 1, wherein the compound is SR-4370, SR-3558, SR-4369, SR-4372, or SR-4373.

Aspect 18: The method of any one of Aspects 1-17, wherein the compound promotes degradation of rest compressor 1 (RCOR1), lysine-specific histone demethylase 1A (LSD1), histone deacetylase 1 (HDAC1), histone deacetylase 2 (HDAC2), or any combination thereof.

Aspect 19: The method in any one of Aspects 1-18, wherein the subject has cancer.

Aspect 20: The method in any one of Aspects 1-18, wherein the subject has breast cancer, prostate cancer, colon cancer, lung cancer, liver cancer, brain tumors, melanomas, Merkel cell carcinomas, leukemias, lymphomas, ovarian cancer, pediatric cancer, hematological disorders, liver fibrosis, infectious diseases, neurodegenerative diseases such as Alzheimer's disease, fatty liver disease and type 2 diabetes, inflammatory diseases such as sepsis, rheumatoid arthritis, or COVID19.

Aspect 21: The method in any one of Aspects 1-20, wherein the amount of rest compressor 1 (RCOR1), lysine-specific histone demethylase 1A (LSD1), histone deacetylase 1 (HDAC1), histone deacetylase 2 (HDAC2), or any combination thereof is measured in a subject prior to the administration of the compound.

Aspect 22: The method in any one of Aspects 1-21, wherein the compound is administered as a pharmaceutical composition.

Aspect 23: A method for treating cancer in a subject comprising administering to the subject in need thereof a compound of formula I or the pharmaceutically acceptable salt thereof

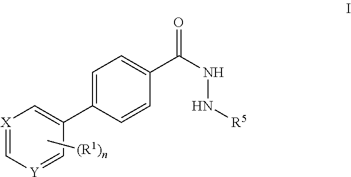

wherein X and Y are independently N or CR²,
wherein R¹ and R² are independently hydrogen, an alkyl group, a halo group, or a carbonyl group,
R⁵ is a C₁ to C₁₀ alkyl group; and
n is an integer from 0 to 3.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Synthesis of SR-4370

A scintillation vial containing a Teflon-coated magnetic stir bar was charged with 2′,3′-difluorobiphenyl-4-carbohydrazide (50 mg, 0.25 mmol). 1 mL of methanol, 22 μL of butyraldehyde (1 eq, 0.25 mmol) and catalytic p-toluenesulfonic acid were subsequently added to the vial. The resultant solution was allowed to stir for 12 h at room temperature. The reaction mixture was then acidified to approximately pH 5 through the addition of 4M HCl in dioxane. Sodium cyanoborohydride (19 mg, 1.2 eq, 0.31 mmol) was added and the reaction mixture was allowed to stir for 3 h at room temperature. The reaction mixture was then concentrated in vacuo. Water was added to the resultant residue followed by diethyl ether. The aqueous layer was extracted three times with diethyl ether. The organic layers were combined, dried and concentrated. The resultant white solid was further purified by flash chromatography utilizing a solvent gradient from 0-65% EtOAc in hexanes.

### SR-4370 Analogs Exhibit Molecular Glue Function to Degrade RCOR1, LSD1, HDAC1 and HDAC2 in a Neddylation and Proteasome-Dependent Manner

MCF7 cells were exposed to DMSO or the indicated compounds (10 μM) for 6 hours. Cells were pretreated with the proteasomal inhibitor MG132 (MG, 3 μM) or the neddylation inhibitor MLN4924 (MLN, 1 μM) for 2 hours. Cells were then lysed and subjected to Western blotting with antibodies against the indicated proteins. The levels of RCOR1 were normalized against that of tubulin and shown as % of DMSO control.

It was unexpectedly found that a subset of compounds with structures similar to SR-4370 (FIG. 1B) markedly downregulated RCOR1, LSD1, HDAC1 and HDAC2 (FIG. 1A). Among these compounds, there is a clear structure-activity relationship. Only SR-4370 and its close structural analogs with a properly positioned biphenyl motif (SR-3558, SR-4369 and SR-4372) can degrade RCOR1, LSD1, HDAC1 and HDAC2 (FIG. 1A). Other compounds without this motif such as UF010 are potent HDACi but is completely inactive to degrade RCOR1, LSD1, HDAC1 and HDAC2 (FIG. 1A).

### SR-4370 Specifically and Potently Degrades RCOR1, LSD1, HDAC1 and HDAC2 in a Neddylation and Proteasome-Dependent Manner

MCF7 cells were exposed to DMSO, SR-4370 and other compounds at the indicated concentrations for 14 hours. Cells were pretreated with the proteasomal inhibitor MG132 (MG, 3 μM) or the neddylation inhibitor MLN4924 (MLN, 1 μM) for 2 hours. Cells were then lysed and subjected to Western blotting with antibodies against the indicated proteins. The levels of RCOR1 and LSD1 were normalized against that of tubulin and shown as % of DMSO control.

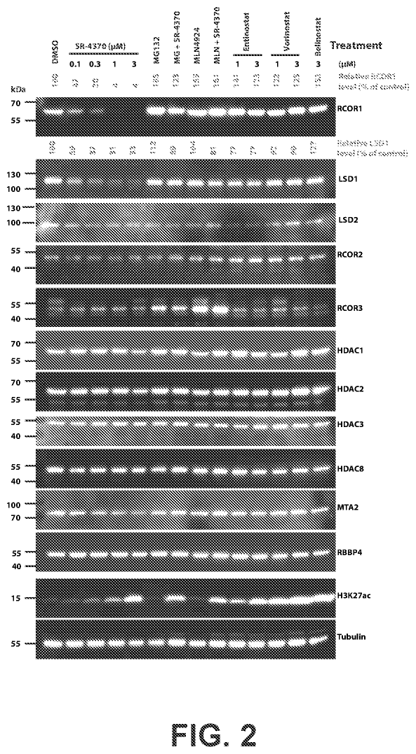

SR-4370 induced potent and dose-dependent degradation of RCOR1, LSD1, HDAC1 and HDAC2 (the DC₅₀ or the concentration at which 50% of the target protein is degraded for RCOR1 is <100 nM), largely without affecting RCOR2 or 3 in MCF7 (FIG. 2), T47D and MDA-MB-231 BC cell lines (data not shown). The downregulation of RCOR1 and LSD1 was completely reversed by inhibition of Cullin neddylation (MLN4924) and inhibition of the proteasome (MG132), indicating that SR-4370 triggers RCOR1 and LSD1 degradation through a Cullin-RING E3 ubiquitin ligase (CRL)-mediated ubiquitination and the proteasome. Other HDACi including class I HDAC-selective entinostat (also known as MS-275) and the FDA-approved vorinostat (SAHA) and belinostat (PXD-101) notably increased the protein level of RCOR1. Entinostat and vorinostat slightly reduced LSD1 level while belinostat moderately increased LSD1 level (FIG. 2).

SR-4370 potently and specifically degrades RCOR1, LSD1, HDAC1 and HDAC2 (FIG. 2). The levels of HDAC1, 2 or MTA2 (an HDAC1/2-binding core subunit of the NuRD complex) were only moderately reduced at a higher concentration of SR-4370. However, RBBP4, a core subunit of the NuRD and the SIN3 complex, was largely unaffected (FIGS. 1 and 2). SR-4370 also did not affect the protein levels of HDAC3 or HDAC8, two other members of class HDACs. LSD2, a histone lysine demethylase related to LSD1, was largely unaffected by SR-4370 (FIG. 2), which is expected as LSD2 does not bind to RCOR1 and HDACs. These data show that SR-4370 specifically targets RCOR1 and LSD1 in the CoREST complex for degradation, sparing other HDAC1/2-containing complexes.

### SR-4370 is Efficacious Against Breast Cancer In Vivo without a Superior Safety Profile

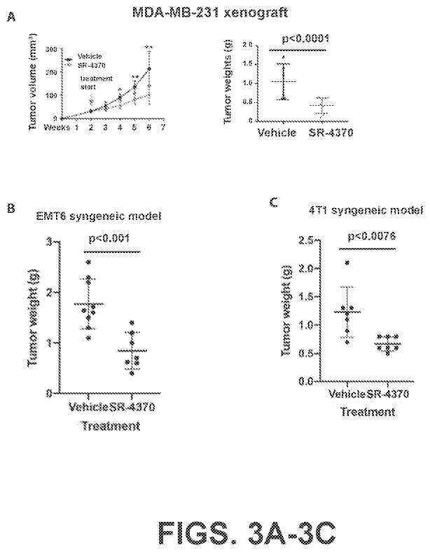

Female athymic mice bearing the MDA-MB-231 xenograft tumors were dosed with vehicle or SR-4370 (FIG. 3A). Tumor volumes and final tumor weights are plotted (average±SD). *: p<0.05, **: p<0.01 (FIGS. 3B and 3C). Female BALB/c mice bearing the orthotopic EMT6 (FIG. 3B) and 4T1 (FIG. 3C) mouse breast tumors were dosed with SR-4370 as in (FIG. 3A). At the endpoint, tumors were removed from euthanized mice and weighed. The tumor weight plots are shown (average±SD). The p values were calculated based on t test.

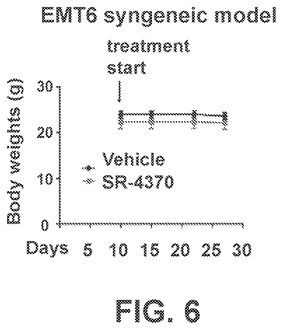

Benzoylhydrazide compounds are less cytotoxic than other known HDACi. In vivo tolerability of UF010, the first identified compound of the benzoylhydrazide HDACi class, was evaluated in mice. Oral dosing of UF010 up to 80 mg/kg was completely tolerated without any signs of toxicity or loss of body weight, indicating that its maximal tolerated dose (MTD) exceeds 80 mg/kg. SR-4370 up to 50 mg/kg was completely tolerated by mice in an in vivo efficacy study (FIG. 3). The body weights of tumor-bearing mice were not affected during the treatment period, indicating that SR-4370 is safe for in vivo use (FIG. 6). Blood chemistry tests of tumor-bearing mice at the endpoint indicate that the serum concentrations of various analytes were within reference ranges for mice after dosing with SR-4370 (Table 1). There were no signs of liver or kidney toxicities associated with these treatments. We conducted pilot experiments to evaluate PK parameters of UF010. As with the MTD experiment, mice were dosed orally at 80 mg/kg. Plasma samples were collected at various time points and the amount of UF010 was determined. As shown in Table 2, UF010 exhibits better plasma drug exposure (AUC) and (Cₘₐₓ) than the aminobenzamide HDACi analog 4b⁷⁴. These data also indicate that UF010 is bioavailable orally. UF010 and the aminobenzamides exhibit similar, relatively short half-life values in rodents. However, even though its stability is less than optimal, the t_{1/2} of UF010 is comparable to a number of cancer pharmaceutics, such as the FDA-approved HDACi belinostat, which has a t_{1/2} of 1 to 2 hr (filing data available at the FDA website). Importantly, the high tolerability of benzoylhydrazide HDACi indicates that desirable therapeutic efficacy may be achieved by higher dosages and more frequent dosing.

TABLE 1
Blood chemistry tests in tumor-bearing athymic mice (average ± SD, n = 6)

| Analytes | Vehicle | SR-4370 | Ref ranges |
|---|---|---|---|
| Total protein (g/dL) | 5.17 (±0.052) | 4.95 (±0.217) | 4.5-6.5 # |
| BUN (mg/dL) | 24.33 (±3.14) | 22.0 (±3.16) | 10-33 # |
| Calcium (mg/dL) | 9.22 (±0.17) | 8.45 (±0.85) | 8.0-15.5 # |
| Cholesterol (mg/dL) | 106.5 (±11.11) | 113.0 (±15.92) | 100-162 ⁷⁵ |
| Chloride (mmol/L) | 110.0 (±2.16) | 110.0 (±2.17) | 90-110 # |
| Glucose (mg/dL) | 177.5 (±45.81) | 221.17 (±60.6) | 190-290 (non-fasting ^{75, 76}) |
| Potassium (mmol/L) | 7.5 (±1.13) | 8.75 (±1.82) | 6.0-8.3 *⁷⁷ |
| Sodium (mmol/L) | 149.0 (±2.16) | 147.33 (±1.51) | 140-160 # |
| Phosphorus (mg/dL) | 6.62 (±1.88) | 9.73 (±2.69) | 4.2-8.5 # |
| Total bilirubin (mg/dL) | 0.27 (±0.082) | 0.25 (±0.054) | 0-1.0 # |
| Creatinine (mg/dL) | 0.1 (±0.075) | 0.1 (±0.044) | 0.06-0.16 ⁷⁸ |
| (ALP, U/L) | 51 (±8.92) | 49.5 (±8.78) | 15-45 # |
| | | | 80-148 ⁷⁹ |
| ALT (U/L) | 58.40 (±23.79) | 71.60 (±14.06) | 22-40 ⁷⁹ |

# University of Pennsylvania Rodent Reference Ranges (available online);
* Methods of euthanasia can affect serum potassium levels in mice ⁷⁷.

TABLE 2
In vivo drug properties of UF010 and benzamide analogs

| | |
|---|---|
| PK | Benzamide analogs |

| parameter ^{a} | UF010 | 4b ^{c} | Ent ^{d} |
|---|---|---|---|
| AUC nM hr | 6070 | 1080 | NA |
| Cₘₐₓ nM | 5952 | 1130 | NA |
| Tₘₐₓ hr | 0.25 | 0.25 | 0.5 |
| T_{1/2} hr | 1.24 | 2.76 | ~1 |
| MRT | 39.3 | NA | NA |
| MTD ^{b} mg/kg | >80 | NA | 3.1 |

^{a} UF010 (80 mg/kg) and 4b (50 mg/kg) were dosed orally.
^{b} MTD was determined in mouse (UF010) or rat (entinostat).
^{c} Data obtained with mice; ref ⁷⁴.
^{d} Data from ref ⁸⁰.

In summary, the in vivo studies show that SR-4370 is safe and has potent anticancer effects in multiple preclinical BC models.

### SR-4370 is More Potent than UF010 to Impair Breast Cancer Cell Growth

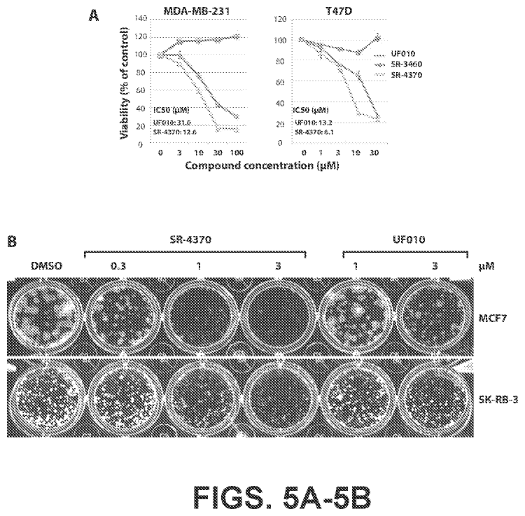

MDA-MB-231 and T47D cells were treated with the indicated compounds for four days (FIG. 5A). Viability assays were done using CellTiter-Glo. Shown are normalized values vs. vehicle±SEM (n=3). IC50 values are calculated using Prism 6. Note that SR-3460 has no HDACi activity. Cells were seeded and then continuously treated with SR-4370 or UF010 at the indicated concentrations for 15 days. Colonies were fixed and stained (FIG. 5B). Compared to UF010, SR-4370 exhibited more potent cytotoxic effects on BC cell lines in short-term or long-term treatment (FIG. 5A). Of note, SR-3460, a structural analog of SR-4370, does not inhibit HDACs, and also did not inhibit BC cell growth (FIG. 5A).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1 Ali, S., Buluwela, L. & Coombes, R. C. Antiestrogens and their therapeutic applications in breast cancer and other diseases. Annual review of medicine 62, 217-232, doi:10.1146/annurev-med-052209-100305 (2011).
2 Jordan, V. C. A century of deciphering the control mechanisms of sex steroid action in breast and prostate cancer: the origins of targeted therapy and chemoprevention. Cancer Res 69, 1243-1254, doi: 10.1158/0008-5472.CAN-09-0029 (2009).
3 Giuliano, M., Schifp, R., Osborne, C. K. & Trivedi, M. V. Biological mechanisms and clinical implications of endocrine resistance in breast cancer. Breast 20 Suppl 3, S42-49, doi: 10.1016/S0960-9776(11)70293-4 (2011).
4 Osborne, C. K. & Schiff, R. Mechanisms of endocrine resistance in breast cancer. Annual review of medicine 62, 233-247, doi: 10.1146/annurev-med-070909-182917 (2011).
5 Droog, M., Beelen, K., Linn, S. & Zwart, W. Tamoxifen resistance: from bench to bedside. European journal of pharmacology 717, 47-57, doi: 10.1016/j.ejphar.2012.11.071 (2013).
6 Nahta, R., Yu, D., Hung, M. C., Hortobagyi, G. N. & Esteva, F. J. Mechanisms of disease: understanding resistance to HER2-targeted therapy in human breast cancer. Nature clinical practice. Oncology 3, 269-280, doi: 10.1038/ncponc0509 (2006).
7 Hudis, C. A. & Gianni, L. Triple-negative breast cancer: an unmet medical need. The oncologist 16 Suppl 1, 1-11, doi: 10.1634/theoncologist.2011-S1-01 (2011).
8 Andre, F. & Zielinski, C. C. Optimal strategies for the treatment of metastatic triple-negative breast cancer with currently approved agents. Ann Oncol 23 Suppl 6, vi46-51, doi: 10.1093/annonc/mds195 (2012).
9 O'Shaughnessy, J. et al. Iniparib plus chemotherapy in metastatic triple-negative breast cancer. N Engl J Med 364, 205-214, doi: 10.1056/NEJMoa1011418 (2011).
10 Rouzier, R. et al. Breast cancer molecular subtypes respond differently to preoperative chemotherapy. Clin Cancer Res 11, 5678-5685, doi: 10.1158/1078-0432.CCR-04-2421 (2005).
11 Parker, J. S. et al. Supervised risk predictor of breast cancer based on intrinsic subtypes. J Clin Oncol 27, 1160-1167, doi: 10.1200/JCO.2008.18.1370 (2009).
12 Muller, B. M. et al. Differential expression of histone deacetylases HDAC1, 2 and 3 in human breast cancer—overexpression of HDAC2 and HDAC3 is associated with clinicopathological indicators of disease progression. BMC Cancer 13, 215, doi:10.1186/1471-2407-13-215 (2013).
13 Kwak, S. M. et al. EGFR-c-Src-Mediated HDAC3 Phosphorylation Exacerbates Invasion of Breast Cancer Cells. Cells 8, doi: 10.3390/cells8080930 (2019).
14 Cui, Z., Xie, M., Wu, Z. & Shi, Y. Relationship Between Histone Deacetylase 3 (HDAC3) and Breast Cancer. Med Sci Monit 24, 2456-2464, doi: 10.12659/msm.906576 (2018).
15 Tang, X. et al. HDAC8 cooperates with SMAD3/4 complex to suppress SIRT7 and promote cell survival and migration. Nucleic Acids Res, doi: 10.1093/nar/gkaa039 (2020).
16 Caslini, C., Hong, S., Ban, Y. J., Chen, X. S. & Ince, T. A. HDAC7 regulates histone 3 lysine 27 acetylation and transcriptional activity at super-enhancer-associated genes in breast cancer stem cells. Oncogene 38, 6599-6614, doi: 10.1038/s41388-019-0897-0 (2019).
17 Gryder, B. E. et al. Chemical genomics reveals histone deacetylases are required for core regulatory transcription. Nat Commun 10, 3004, doi: 10.1038/s41467-019-11046-7 (2019).
18 Delcuve, G. P., Khan, D. H. & Davie, J. R. Roles of histone deacetylases in epigenetic regulation: emerging paradigms from studies with inhibitors. Clinical epigenetics 4, 5, doi:10.1186/1868-7083-4-5 (2012).
19 Munster, P. et al. Clinical and biological effects of valproic acid as a histone deacetylase inhibitor on tumor and surrogate tissues: phase I/II trial of valproic acid and epirubicin/FEC. Clin Cancer Res 15, 2488-2496, doi: 10.1158/1078-0432.CCR-08-1930 (2009).
20 Munster, P. N. et al. A phase II study of the histone deacetylase inhibitor vorinostat combined with tamoxifen for the treatment of patients with hormone therapy-resistant breast cancer. Br J Cancer 104, 1828-1835, doi: 10.1038/bjc.2011.156 (2011).
21 Munster, P. N. et al. Phase I trial of vorinostat and doxorubicin in solid tumours: histone deacetylase 2 expression as a predictive marker. Br J Cancer 101, 1044-1050, doi: 10.1038/sj.bjc.6605293 (2009).
22 Millard, C. J., Watson, P. J., Fairall, L. & Schwabe, J. W. Targeting Class I Histone Deacetylases in a “Complex” Environment. Trends Pharmacol Sci 38, 363-377, doi: 10.1016/j.tips.2016.12.006 (2017).
23 You, A., Tong, J. K., Grozinger, C. M. & Schreiber, S. L. CoREST is an integral component of the CoREST-human histone deacetylase complex. Proc Natl Acad Sci USA 98, 1454-1458, doi:10.1073/pnas.98.4.1454 (2001).
24 Shi, Y. et al. Histone demethylation mediated by the nuclear amine oxidase homolog LSD1. Cell 119, 941-953, doi: 10.1016/j.cell.2004.12.012 (2004).
25 Song, Y. et al. Mechanism of Crosstalk between the LSD1 Demethylase and HDAC1 Deacetylase in the CoREST Complex. Cell Rep 30, 2699-2711 e2698, doi: 10.1016/j.celrep.2020.01.091 (2020).
26 Laurent, B. et al. A specific LSD1/KDM1A isoform regulates neuronal differentiation through H3K9 demethylation. Mol Cell 57, 957-970, doi: 10.1016/j.molcel.2015.01.010 (2015).
27 Wu, M. et al. Lysine-14 acetylation of histone H3 in chromatin confers resistance to the deacetylase and demethylase activities of an epigenetic silencing complex. Elife 7, doi: 10.7554/eLife.37231 (2018).
28 Kalin, J. H. et al. Targeting the CoREST complex with dual histone deacetylase and demethylase inhibitors. Nat Commun 9, 53, doi: 10.1038/s41467-017-02242-4 (2018).
29 Maksour, S., Ooi, L. & Dottori, M. More than a Corepressor: The Role of CoREST Proteins in Neurodevelopment. eNeuro 7, doi: 10.1523/ENEURO.0337-19.2020 (2020).
30 Wang, Z. et al. Genome-wide mapping of HATs and HDACs reveals distinct functions in active and inactive genes. Cell 138, 1019-1031, doi:10.1016/j.cell.2009.06.049 (2009).
31 Gryder, B. E. et al. Histone hyperacetylation disrupts core gene regulatory architecture in rhabdomyosarcoma. Nat Genet 51, 1714-1722, doi: 10.1038/s41588-019-0534-4 (2019).
32 Clark, E. A. et al. GR and LSD1/KDM1A-Targeted Gene Activation Requires Selective H3K4me2 Demethylation at Enhancers. 27, 3522-3532 Cell Rep e3523, doi: 10.1016/j.celrep.2019.05.062 (2019).
33 Wu, Y. et al. The deubiquitinase USP28 stabilizes LSD1 and confers stem-cell-like traits to breast cancer cells. Cell Rep 5, 224-236, doi: 10.1016/j.celrep.2013.08.030 (2013).
34 Verigos, J. et al. The Histone Demethylase LSD1/KappaDM1A Mediates Chemoresistance in Breast Cancer via Regulation of a Stem Cell Program. Cancers (Basel) 11, doi:10.3390/cancers11101585 (2019).
35 Qin, Y. et al. Inhibition of histone lysine-specific demethylase 1 elicits breast tumor immunity and enhances antitumor efficacy of immune checkpoint blockade. Oncogene 38, 390-405, doi: 10.1038/s41388-018-0451-5 (2019).
36 Nagasawa, S. et al. LSD1 overexpression is associated with poor prognosis in basal-like breast cancer, and sensitivity to PARP inhibition. PLoS One 10, e0118002, doi:10.1371/journal.pone.0118002 (2015).
37 Mazumdar, S. et al. CoREST1 promotes tumor formation and tumor stroma interactions in a mouse model of breast cancer. PLoS One 10, e0121281, doi:10.1371/journal.pone.0121281 (2015).
38 Miller, S. A. et al. Lysine-Specific Demethylase 1 Mediates AKT Activity and Promotes Epithelial-to-Mesenchymal Transition in PIK3CA-Mutant Colorectal Cancer. Mol Cancer Res 18, 264-277, doi: 10.1158/1541-7786.MCR-19-0748 (2020).
39 Soucy, T. A. et al. An inhibitor of NEDD8-activating enzyme as a new approach to treat cancer. Nature 458, 732-736, doi: 10.1038/nature07884 (2009).
40 Majello, B., Gorini, F., Sacca, C. D. & Amente, S. Expanding the Role of the Histone Lysine-Specific Demethylase LSD1 in Cancer. Cancers (Basel) 11, doi: 10.3390/cancers11030324 (2019).
41 Perillo, B. et al. DNA oxidation as triggered by H3K9me2 demethylation drives estrogen-induced gene expression. Science 319, 202-206, doi: 10.1126/science. 1147674 (2008).
42 Wang, Y. et al. LSD1 is a subunit of the NuRD complex and targets the metastasis programs in breast cancer. Cell 138, 660-672, doi: 10.1016/j.cell.2009.05.050 (2009).
43 Vasilatos, S. N. et al. Crosstalk between lysine-specific demethylase 1 (LSD1) and histone deacetylases mediates antineoplastic efficacy of HDAC inhibitors in human breast cancer cells. Carcinogenesis 34, 1196-1207, doi: 10.1093/carcin/bgt033 (2013).
44 Yang, Y. et al. LSD1 coordinates with the SIN3A/HDAC complex and maintains sensitivity to chemotherapy in breast cancer. J Mol Cell Biol 10, 285-301, doi:10.1093/jmcb/mjy021 (2018).
45 Metzger, E. et al. LSD1 demethylates repressive histone marks to promote androgen-receptor-dependent transcription. Nature 437, 436-439, doi: 10.1038/nature04020 (2005).
46 Sehrawat, A. et al. LSD1 activates a lethal prostate cancer gene network independently of its demethylase function. Proc Natl Acad Sci USA 115, E4179-E4188, doi: 10.1073/pnas. 1719168115 (2018).
47 Mohammad, H. P. et al. A DNA Hypomethylation Signature Predicts Antitumor Activity of LSD1 Inhibitors in SCLC. Cancer Cell 28, 57-69, doi: 10.1016/j.ccell.2015.06.002 (2015).
48 Takagi, S. et al. LSD1 Inhibitor T-3775440 Inhibits SCLC Cell Proliferation by Disrupting LSD1 Interactions with SNAG Domain Proteins INSM1 and GFI1B. Cancer Res 77, 4652-4662, doi: 10.1158/0008-5472.CAN-16-3502 (2017).
49 Sakamoto, A. et al. Lysine Demethylase LSD1 Coordinates Glycolytic and Mitochondrial Metabolism in Hepatocellular Carcinoma Cells. Cancer Res 75, 1445-1456, doi: 10.1158/0008-5472.CAN-14-1560 (2015).
50 Leiendecker, L. et al. LSD1 inhibition induces differentiation and cell death in Merkel cell carcinoma. EMBO Mol Med, e12525, doi:10.15252/emmm.202012525 (2020).
51 Ishikawa, Y. et al. A Novel LSD1 Inhibitor T-3775440 Disrupts GFI1B-Containing Complex Leading to Transdifferentiation and Impaired Growth of AML Cells. Mol Cancer Ther 16, 273-284, doi:10.1158/1535-7163.Mct-16-0471 (2017).
52 Maes, T. et al. ORY-1001, a Potent and Selective Covalent KDM1A Inhibitor, for the Treatment of Acute Leukemia. Cancer Cell 33, 495-511 e412, doi: 10.1016/j.ccell.2018.02.002 (2018).
53 Maiques-Diaz, A. et al. Enhancer Activation by Pharmacologic Displacement of LSD1 from GFI1 Induces Differentiation in Acute Myeloid Leukemia. Cell Rep 22, 3641-3659, doi: 10.1016/j.celrep.2018.03.012 (2018).
54 Johnston, G. et al. Nascent transcript and single-cell RNA-seq analysis defines the mechanism of action of the LSD1 inhibitor INCB059872 in myeloid leukemia. Gene 752, 144758, doi:10.1016/j.gene.2020.144758 (2020).
55 Ravasio, R. et al. Targeting the scaffolding role of LSD1 (KDM1A) poises acute myeloid leukemia cells for retinoic acid-induced differentiation. Sci Adv 6, eaax2746, doi: 10.1126/sciadv.aax2746 (2020).
56 Salamero, O. et al. First-in-Human Phase I Study of ladademstat (ORY-1001): A First-in-Class Lysine-Specific Histone Demethylase 1A Inhibitor, in Relapsed or Refractory Acute Myeloid Leukemia. J Clin Oncol 38, 4260-4273, doi: 10.1200/JCO. 19.03250 (2020).
57 Tayari, M. M. et al. Clinical Responsiveness to All-trans Retinoic Acid Is Potentiated by LSD1 Inhibition and Associated with a Quiescent Transcriptome in Myeloid Malignancies. Clin Cancer Res 27, 1893-1903, doi: 10.1158/1078-0432.CCR-20-4054 (2021).
58 Soldi, R. et al. The novel reversible LSD1 inhibitor SP-2577 promotes anti-tumor immunity in SWItch/Sucrose-NonFermentable (SWI/SNF) complex mutated ovarian cancer. PLoS One 15, e0235705, doi: 10.1371/journal.pone.0235705 (2020).
59 Gupta, S. et al. Reversible LSD1 inhibition with HCl-2509 induces the p53 gene expression signature and disrupts the MYCN signature in high-risk neuroblastoma cells. Oncotarget 9, 9907-9924, doi: 10.18632/oncotarget.24035 (2018).
60 Anastas, J. N. et al. Re-programing Chromatin with a Bifunctional LSD1/HDAC Inhibitor Induces Therapeutic Differentiation in DIPG. Cancer Cell 36, 528-544 e510, doi:10.1016/j.ccell.2019.09.005 (2019).
61 Huang, Y., Vasilatos, S. N., Boric, L., Shaw, P. G. & Davidson, N. E. Inhibitors of histone demethylation and histone deacetylation cooperate in regulating gene expression and inhibiting growth in human breast cancer cells. Breast Cancer Res Treat 131, 777-789, doi: 10.1007/s10549-011-1480-8 (2012).
62 Bennesch, M. A., Segala, G., Wider, D. & Picard, D. LSD1 engages a corepressor complex for the activation of the estrogen receptor alpha by estrogen and CAMP. Nucleic Acids Res 44, 8655-8670, doi:10.1093/nar/gkw522 (2016).
63 Gao, S. et al. Chromatin binding of FOXA1 is promoted by LSD1-mediated demethylation in prostate cancer. Nat Genet, doi: 10.1038/s41588-020-0681-7 (2020).
64 Sheng, W. et al. LSD1 Ablation Stimulates Anti-tumor Immunity and Enables Checkpoint Blockade. Cell 174, 549-563 e519, doi: 10.1016/j.cell.2018.05.052 (2018).
65 Tu, W. J. et al. Targeting Nuclear LSD1 to Reprogram Cancer Cells and Reinvigorate Exhausted T Cells via a Novel LSD1-EOMES Switch. Front Immunol 11, 1228, doi: 10.3389/fimmu.2020.01228 (2020).
66 Xiong, Y. et al. Inhibiting the coregulator CoREST impairs Foxp3+ Treg function and promotes antitumor immunity. J Clin Invest 130, 1830-1842, doi: 10.1172/JCI131375 (2020).
67 Ambrosio, S., Ballabio, A. & Majello, B. Histone methyl-transferases and demethylases in the autophagy regulatory network: the emerging role of KDM1A/LSD1 demethylase. Autophagy 15, 187-196, doi: 10.1080/15548627.2018.1520546 (2019).
68 Liu, Y. et al. Hepatic Slug epigenetically promotes liver lipogenesis, fatty liver disease, and type 2 diabetes. J Clin Invest 130, 2992-3004, doi: 10.1172/JCI128073 (2020).
69 Kim, D., Kim, K. I. & Baek, S. H. Roles of lysine-specific demethylase 1 (LSD1) in homeostasis and diseases. J Biomed Sci 28, 41, doi:10.1186/s12929-021-00737-3 (2021).
70 Kim, D. et al. PKCalpha-LSD1-NF-kappaB-Signaling Cascade Is Crucial for Epigenetic Control of the Inflammatory Response. Mol Cell 69, 398-411 e396, doi:10.1016/j.molcel.2018.01.002 (2018).
71 Hong, K. S. et al. GSK-LSD1, an LSD1 inhibitor, quashes SARS-COV-2-triggered cytokine release syndrome in-vitro. Signal Transduct Target Ther 5, 267, doi: 10.1038/s41392-020-00391-5 (2020).
72 Chagraoui, J. et al. UM171 Preserves Epigenetic Marks that Are Reduced in Ex Vivo Culture of Human HSCs via Potentiation of the CLR3-KBTBD4 Complex. Cell stem cell 28, 48-62 e46, doi: 10.1016/j.stem.2020.12.002 (2021).
73 Subramaniam, A. et al. Lysine-specific demethylase 1A restricts ex vivo propagation of human HSCs and is a target of UM171. Blood 136, 2151-2161, doi:10.1182/blood.2020005827 (2020).
74 Beconi, M. et al. Oral administration of the pimelic diphenylamide HDAC inhibitor HDACi 4b is unsuitable for chronic inhibition of HDAC activity in the CNS in vivo. PLoS One 7, e44498, doi: 10.1371/journal.pone.0044498 (2012).
75 Fernandez, I., Pena, A., Del Teso, N., Perez, V. & Rodriguez-Cuesta, J. Clinical biochemistry parameters in C57BL/6J mice after blood collection from the submandibular vein and retroorbital plexus. J Am Assoc Lab Anim Sci 49, 202-206 (2010).
76 Togashi, Y. et al. Evaluation of the appropriateness of using glucometers for measuring the blood glucose levels in mice. Sci Rep 6, 25465, doi: 10.1038/srep25465 (2016).
77 Traslavina, R. P. et al. Euthanasia by CO(2) inhalation affects potassium levels in mice. J Am Assoc Lab Anim Sci 49, 316-322 (2010).
78 Palm, M. & Lundblad, A. Creatinine concentration in plasma from dog, rat, and mouse: a comparison of 3 different methods. Vet Clin Pathol 34, 232-236 (2005).
79 Otto, G. P. et al. Clinical Chemistry Reference Intervals for C57BL/6J, C57BL/6N, and C3HeB/FeJ Mice (Mus musculus). J Am Assoc Lab Anim Sci 55, 375-386 (2016).
80 Ryan, Q. C. et al. Phase I and pharmacokinetic study of MS-275, a histone deacetylase inhibitor, in patients with advanced and refractory solid tumors or lymphoma. J Clin Oncol 23, 3912-3922, doi: 10.1200/JCO.2005.02.188 (2005).

1. A method for reducing the amount of rest compressor 1 (RCOR1), lysine-specific histone demethylase 1A (LSD1), histone deacetylase 1 (HDAC1), histone deacetylase 2 (HDAC2), or any combination thereof in a subject comprising administering to the subject a compound of formula I or the pharmaceutically acceptable salt thereof 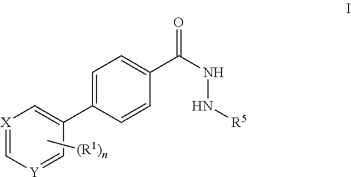
wherein X and Y are independently N or CR²,
wherein R¹ and R² are independently hydrogen, an alkyl group, a halo group, an ester group,
R⁵ is a C₁ to C₁₀ alkyl group;
and
n is an integer from 0 to 3.

2. The method of claim 1, wherein X and Y are each N.

3. The method of claim 2, wherein n is 0.

4. The method of claim 1, wherein R⁵ is a butyl group.

5. The method of claim 1, wherein X and Y are each CR².

6. The method of claim 1, wherein X is CF.

7. The method of claim 1, wherein X is CH.

8. The method of claim 1, wherein X is C—C(O)OR³, wherein R³ is an alkyl group.

9. The method of claim 5, wherein Y is CH.

10. The method of claim 5, wherein Y is CR⁴, wherein R⁴ is an alkyl group.

11. The method of claim 5, wherein n is 0.

12. The method of claim 5, wherein R⁵ is a butyl group.

13. The method of claim 1, wherein X is CF and Y is CH or CR⁴, wherein R⁴ is an alkyl group.

14. The method of claim 1, wherein the compound has the formula II 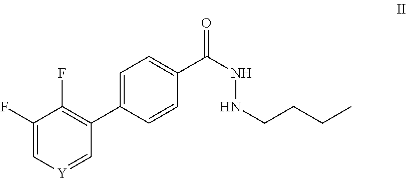
wherein Y is CH or CR⁴, wherein R⁴ is an alkyl group.

15. The method of claim 1, wherein X is CF, Y is CH, R¹ is fluoro, and n is 1.

16. The method of claim 1, wherein the compound has the formula III 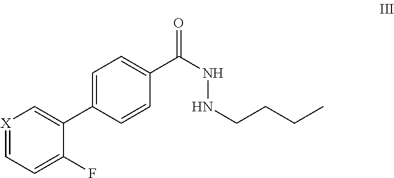
wherein X is C—C(O)OR³, wherein R³ is an alkyl group.

17. The method of claim 1, wherein the compound is SR-4370, SR-3558, SR-4369, SR-4372, or SR-4373.

18. (canceled)

19. The method of claim 1, wherein the subject has cancer.

20. The method of claim 1, wherein the subject has breast cancer, prostate cancer, a brain tumor, colon cancer, lung cancer, liver cancer, melanomas, Merkel cell carcinomas, leukemias, lymphomas, ovarian cancer, or pediatric cancer.

21. The method of claim 1, wherein the subject has a hematological disorder, liver fibrosis, an infectious disease, a neurodegenerative disease, a fatty liver disease, type 2 diabetes, an inflammatory disease, rheumatoid arthritis, or COVID19.

22. The method of claim 1, wherein the amount of rest compressor 1 (RCOR1), lysine-specific histone demethylase 1A (LSD1), histone deacetylase 1 (HDAC1), histone deacetylase 2 (HDAC2), or any combination thereof is measured in a subject prior to the administration of the compound.

23. The method of claim 1, wherein the compound is administered as a pharmaceutical composition.

24. A method for treating cancer in a subject comprising administering to the subject in need thereof a compound of formula I or the pharmaceutically acceptable salt thereof 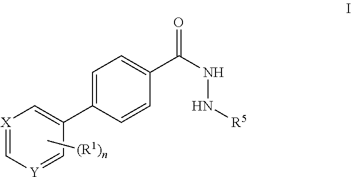
wherein X and Y are independently N or CR²,
wherein R¹ and R² are independently hydrogen, an alkyl group, a halo group, or a carbonyl group,
R⁵ is a C₁ to C₁₀ alkyl group; and
n is an integer from 0 to 3.
